(19) United States
(12) Patent Application Publication
(10) Pub. No.: US 2009/0192332 A1
(43) Pub. Date: Jul. 30, 2009

(54) Method for Preparing Isoprenyl Cysteine Compounds and Analogs Thereof

(76) Inventors: Keshava Rapole, Edison, NJ (US); Jeffry B. Stock, Princeton, NJ (US); Michael Voronkov, Pennington, NJ (US)

Correspondence Address: CHOATE, HALL & STEWART LLP, TWO INTERNATIONAL PLACE, BOSTON, MA, 02110, US

(21) Appl. No.: 12/358,712

(22) Filed: Jan. 23, 2009

Related U.S. Application Data
(60) Provisional application No. 61/062,263, filed on Jan. 24, 2008.
(60) Provisional application No. 61/068,920, filed on Mar. 11, 2008.

(51) Int. Cl. C07C 51/42; C07C 51/347

(52) U.S. Cl. 562/600; 562/598

(57) ABSTRACT

Methods of preparing isoprenyl cysteine compounds by coupling a cysteine compound with an activated (i.e. halogenated) lipid are disclosed. Also disclosed, among other things, are methods of making activated (i.e. halogenated) lipids, and methods of purifying isoprenyl cysteine compounds.

### RELATED APPLICATIONS

This application claims priority to U.S. provisional patent application Ser. No. 61/062,263, filed Jan. 24, 2008, and U.S. provisional patent application Ser. No. 61/068,920, filed Mar. 11, 2008, the entire disclosure of each of which is incorporated herein by reference.

### BACKGROUND

Inflammation often is a bodily response to infection or injury in which cells involved in detoxification and repair are mobilized to the compromised site by inflammatory mediators. The infection or injury can be a result of acute or chronic disease, disorders, conditions or trauma, environmental conditions, or aging. Examples include diseases, disorders, syndromes, conditions and injuries of the cardiovascular, digestive, integumentary, muscular, nervous, reproductive, respiratory and urinary systems, as well as diseases, disorders, syndromes, conditions and injuries of tissue and cartilage such as atherosclerosis, irritable bowel syndrome, psoriasis, tendonitis, Alzheimer's disease and vascular dementia, multiple sclerosis, diabetes, endometriosis, asthma and kidney failure.

N-acetyl-S-farnesyl-L-cysteine (“AFC”), also referred to as N-acetyl-S-trans, trans-farnesyl-L-cysteine, is a signal transduction modulator that has been shown to reduce inflammation in mice. AFC is a polyisoprenyl-protein inhibitor, and has been shown to be a competitive inhibitor of membrane-associated isoprenyl-S-cysteinyl methyltransferase. AFC has also been shown to block some neutrophil, macrophage, and platelet responses in vitro. Treatment of inflammatory diseases or disorders with traditional anti-inflammatory drugs, e.g., corticosteroids and non-steroidal anti-inflammatory drugs (“NSAIDS”) can cause multiple side effects, e.g., appetite and weight gain, excess sweating, high blood pressure, nausea, vomiting, diarrhea, etc. AFC and analogs thereof (i.e., isoprenyl cysteine compounds) are desirable and effective inhibitors of inflammation. See, for example, U.S. Pat. No. 6,372,793; U.S. Pat. No. 5,043,268; U.S. Pat. No. 5,202,456; PCT Publication No. WO05/123103; US Publication No. 2005/0277694; PCT Publication No. WO06/135894; PCT Publication No. WO92/018,465.

### SUMMARY

Among other things, the present invention encompasses the recognition that it would be desirable to develop a method of preparing isoprenyl cysteine compounds with high yield and/or few impurities. For example, the invention encompasses the recognition that there is a need for a method of preparing isoprenyl cysteine compounds that are free of odiferous impurities including acetic acid and/or sulfur-containing impurities, by-products, and starting materials.

Generally, the methods of the present invention provide for increased yields and/or fewer impurities as compared with prior art methods of preparing isoprenyl cysteine compounds. Moreover, methods of the present invention allow for an efficient and high-yield procedure for preparing larger quantities (i.e. 100 grams or more) of isoprenyl cysteine compounds, particularly as compared to using the more dilute reaction conditions described previously by others. Furthermore, methods of the present invention avoid the unwanted presence of odiferous impurities, such as acetic acid, sulfur-containing impurities, sulfur-containing by-products, and sulfur-containing starting materials.

In certain embodiments, the present invention provides a method of making isoprenyl cysteine compounds comprising coupling a cysteine compound with an activated (i.e., halogenated) lipid in the presence of a base and a solvent.

In certain embodiments, an activated (halogenated) lipid is derived from an allylic alcohol. The allylic alcohol can be a substituted or unsubstituted, saturated or unsaturated, C₁₀₋₂₀ allylic alcohol. Particular examples of activated lipids useful in accordance with the present invention include farnesyl bromide, farnesyl chloride, phytyl bromide, phytyl chloride, geranyl bromide, geranyl chloride, geranyl geranyl bromide, or geranyl geranyl chloride.

In certain embodiments, the solvent for the coupling reaction is a non-aqueous solvent. Exemplary non-aqueous solvents useful in the practice of the present invention include propanol, including isopropanol, ethanol, methanol, butanol, including isobutanol, dioxane, dimethoxyethane, bis(2-methoxyethyl)ether, octanol, t-butyl alcohol, tetrahydrofuran (“THF”), and/or combinations thereof.

In certain embodiments, the base can be Na₂CO₃, K₂CO₃, NaHCO₃, KHCO₃, CH₃CO₂Na, CH₃CO₂K, KOH, NaOH, LiOH, Na₂HPO₄, K₂HPO₄, Na₃PO₄, and/or K₃PO₄.

In certain embodiments, the reaction is run at a temperature between room temperature and the boiling point of the solvent.

In certain embodiments, the present invention provides a method of making N-acetyl-S-farnesyl-L-cysteine comprising reacting N-acetyl-cysteine with a halogenated lipid in the presence of an inorganic base in isopropyl alcohol at a temperature of about 40° C. or more. In certain embodiments the temperature is about 80° C.-85° C. In certain embodiments, the temperature is about 80° C. In certain embodiments, the inorganic base is a carbonate or a bicarbonate.

In certain embodiments, the present invention provides a method of making N-acetyl-S-farnesyl-cysteine comprising coupling farnesyl bromide with N-acetyl-cysteine in isopropyl alcohol and a weak base at a temperature of about 80° C.-85° C., followed by purification. In certain embodiments, the temperature is about 80° C.

### DETAILED DESCRIPTION OF CERTAIN EMBODIMENTS

### I. General Methods of Preparation

The present invention provides synthetic methodologies for preparing isoprenyl cysteine compounds comprising coupling a cysteine compound with an activated (i.e. halogenated) lipid in the presence of a base and a solvent. In certain embodiments, the isoprenyl cysteine compound is further purified.

In certain embodiments, isoprenyl cysteine compounds are generally prepared according to Scheme 1 set forth below.

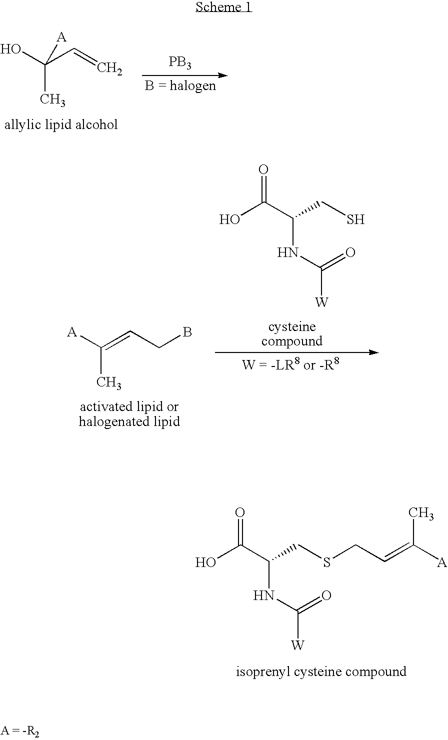

In certain embodiments, isoprenyl cysteine compounds are generally prepared by: (a) coupling an activated lipid with a cysteine compound in a non-aqueous solvent at a molar concentration ranging from about 0.1 M to about 5 M (i.e., 0.1 mol-5 mol per 1 L of solvent) in the presence of 0.5-1.5 equivalents of an inorganic base at a temperature of about 80° C.-85° C. to yield a reaction mixture containing an isoprenyl cysteine compound; and (b) purifying a concentrated reaction mixture containing an isoprenyl cysteine compound comprising steps of: (i) contacting the reaction mixture with acetonitrile wash to remove non-polar impurities from the reaction mixture; and (ii) adjusting pH of the reaction mixture using an aqueous acid, to obtain the isoprenyl cysteine compound with a purity of at least 95%.

In some embodiments, the present invention provides a method of preparing N-acetyl-S-farnesyl-L-cysteine comprising coupling farnesyl bromide with N-acetyl-cysteine in isopropyl alcohol and a weak base at a temperature of about 80° C., followed by purification.

In certain embodiments, N-acetyl-S-farnesyl-L-cysteine is generally prepared by: (a) coupling farnesyl bromide with N-acetyl-cysteine in a non-aqueous solvent at a molar concentration ranging from about 0.1 M to about 5 M in the presence of 0.5-1.5 equivalents of an inorganic base at a temperature of about 80° C.-85° C. to yield a reaction mixture containing N-acetyl-S-farnesyl-L-cysteine; and (b) purifying a concentrated reaction mixture containing N-acetyl-S-farnesyl-L-cysteine comprising steps of: (i) contacting the reaction mixture with acetonitrile wash to remove non-polar impurities from the reaction mixture; and (ii) adjusting pH of the reaction mixture using an aqueous acid, to obtain the N-acetyl-S-farnesyl-L-cysteine with a purity of at least 95%.

In certain embodiments, the isoprenyl cysteine compounds are obtained in a yield of at least 80%. In certain embodiments, the isoprenyl cysteine compound is N-acetyl-S-farnesyl-L-cysteine. In certain embodiments, is N-acetyl-S-farnesyl-L-cysteine is obtained in a yield of at least 80%.

In certain embodiments, N-acetyl-S-farnesyl-L-cysteine (“AFC”) is prepared by coupling an activated (i.e. halogenated) lipid with a cysteine compound. The activated lipid can desirably be produced by reacting PX₃ (where X is a halogen) with an allylic alcohol in the presence of a base and in a non-polar solvent, followed by recovery of the product. In certain embodiments, coupling of a cysteine compound with a halogenated lipid is conducted at elevated temperatures in a polar solvent in the presence of a weak inorganic base. The crude product is then purified, for example, by acidification or conversation to a water-insoluble salt. These steps are discussed in more detail herein and are shown in Scheme 5.

Additional background information and methods of preparation of isoprenyl cysteine compounds have been previously described in U.S. Pat. No. 5,043,268 (Stock et al.), U.S. Pat. No. 5,202,456 (Rando et al.), and U.S. Pat. No. 5,705,528 (Kloog et al.), as well as U.S. patent application Ser. No. 2005/0277694 (Stock et al.) and U.S. patent application Ser. No. 2007/0004803 (Gibbs et al.) each of which is incorporated herein by reference.

a. Activated Lipid Formation

Activated lipids may be purchased from a commercial source and then coupled to the cysteine-containing compound, or can be produced according to known methods. Advantageously, an activated lipid can be prepared by a method disclosed herein, for example, according to Scheme 2 below. In general, this novel approach results in fewer impurities and an overall improved reaction yield.

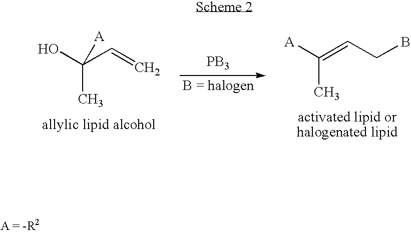

Specifically, a substituted or unsubstituted, saturated or unsaturated, C₁₀₋₂₀ allylic alcohol is dissolved in a suitable solvent with a weak organic base. The resulting solution is maintained at a controlled temperature, ranging from about −20° C. to about 20° C. In certain embodiments, the resulting solution is maintained at a controlled temperature ranging from −10° C. to 20° C. In certain embodiments, the solution is stirred.

Useful C₁₀₋₂₀ allylic alcohols include primary, secondary and tertiary allylic alcohols. In certain embodiments, the allylic alcohol is a terpene or a sesquiterpene. In certain embodiments, the allylic alcohol is farnesol (3,7,11-trimethyl-2,6,10-dodecatrien-1-ol), phytyl ((2E,7R,11R)-3,7,11,15-tetramethyl-2-hexadecen-1-ol), linalool (7-dimethylocta-1,6-dien-3-ol), nerolidol (3,7,11-trimethyll,6,10-dodecatrien-3-ol) or geranyl linalool (3,7-dimethyl-2,6-octadien-1-ol). In certain embodiments, the allylic alcohol is nerolidol, a less costly alternative to farnesol.

In certain embodiments, the organic base has a pK ranging from about 8 to about 12. In certain embodiments, the organic base is triethylamine, ethyldiisopropylamine, or 2,2,6,6-tetramethylpiperidine. In certain embodiments, the organic base is triethylamine. While not intending to be bound by any particular theory of operation, it is believed that the use of an organic base, as opposed to an inorganic base, prevents excessive foaming during manufacturing and/or large scale production and allows for increased reactor load. Moreover, because inorganic bases may clump together or form solid rocks, which could potentially damage a reactor, it is also believed that the use of a weak organic base will avoid such damage. Generally, about 0.05 moles to about 2.0 moles of base are used per mole of allylic alcohol. In certain embodiments, about 0.1 moles to about 0.5 moles of base per mole are used per mole of allylic alcohol.

Useful solvents include any hydrocarbon or substituted hydrocarbon that will not react with the activating (halogenating) agent. In certain embodiments, the solvent has a boiling point between about 30° C. and about 200° C. In certain embodiments, the solvent is toluene, hexane, heptane, pentane, xylene, chlorobenzene, and ether. In certain embodiments, the solvent is toluene.

Next, about 0.3 moles to about 0.5 moles of a halogenating agent, such as, without limitation, PBr₃ or PCl₃, per mole of allylic alcohol is dissolved in the same solvent (in a separate reaction vessel) and added to the allylic alcohol solution. In certain embodiments, the allylic alcohol in solvent is added slowly to the allylic alcohol solution. In certain embodiments the reaction is run under an atmosphere of nitrogen. In certain embodiments the reaction is run under an atmosphere of argon. This reaction is allowed to proceed during the addition of PBr₃ or PCl₃ at a temperature ranging from about −20° C. to about 20° C. The reaction is complete about 30 minutes to about 2 hours after the addition of the halogenating agent is finished. After the reaction is complete, the mixture is warmed to about 20° C. or more. These times and temperatures may be adjusted depending on the halogenation source.

The crude reaction mixture is then washed with water and brine (i.e., water saturated or nearly saturated with NaCl) to remove unreacted starting materials and any other water-soluble impurities. The organic layers are collected and the solvent is evaporated to recover the activated lipid product as an oil. In certain embodiments the solvent is evaporated under reduced pressure.

b. Coupling Activated Lipid to a Cysteine Compound

Regardless of the origin of the activated (halogenated) lipid, the activated lipid is reacted with a cysteine compound to produce a desired isoprenyl cysteine. In certain embodiments, the activated lipid is reacted with a cysteine compound to produce the desired isoprenyl cysteine compound according to Scheme 3 set forth below.

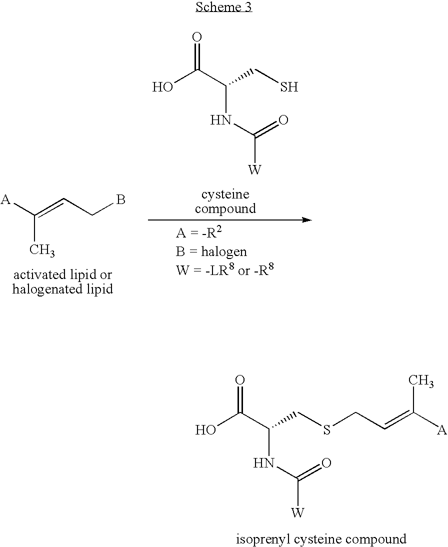

In certain embodiments, isoprenyl cysteine compounds are generally prepared by coupling an activated lipid with a cysteine compound in isopropyl alcohol at a molar concentration ranging from about 0.1 M to about 5 M in the presence of 0.5-1.5 equivalents of an inorganic base at a temperature of about 80° C.-85° C., wherein the isoprenyl cysteine compound has a purity of at least 95%.

In certain embodiments, isoprenyl cysteine compounds are generally prepared by coupling farnesyl bromide with N-acetyl-cysteine in isopropyl alcohol at a molar concentration ranging from about 0.1 M to about 5 M in the presence of 0.5-1.5 equivalents of an inorganic base at a temperature of about 80° C.-85° C., wherein the N-acetyl-S-farnesyl-L-cysteine has a purity of at least 95%.

In certain embodiments, the coupling is performed at a temperature of about 80° C.-85° C. In certain embodiments, the coupling is performed at a temperature of about 80° C.

To accomplish this coupling, a suspension or slurry of a cysteine compound (or a cysteine-containing compound) with a suitable inorganic base is prepared in a non-aqueous solvent. Suitable inorganic bases include Na₂CO₃, K₂CO₃, NaHCO₃, KHCO₃, CH₃CO₂Na, CH₃CO₂K, NaOH, LiOH, KOH, Na₂HPO₄, K₂HPO₄, Na₃PO₄, K₃PO₄. In certain embodiments, the inorganic base is Na₂CO₃. In certain embodiments, the inorganic base is K₂CO₃. Because the inorganic base is not readily soluble in the non-aqueous solvent, the slurry acts like a buffer, and prevents unnecessary hydrolysis and/or decomposition of the starting reagents or products. It is believed that the relative insolubility of the buffer in this system allows it to be consumed more slowly as the reaction progresses and drives the equilibrium toward further dissolution of the buffer. Typically about 1.0 to 4.0 moles of inorganic base are used per mole of cysteine compound. The presence or addition of base in the reaction is necessary to activate the thiol or mercapto moiety for coupling, and also acts as a drying agent to reduce the hydrolysis of the activated lipid.

Alternatively, the cysteine compound may be dissolved in a suitable solvent, including water, and either an organic base or an inorganic base is added to the solution concurrently with the activated lipid at a rate sufficient to maintain the reaction conditions.

Any cysteine compound may be used including the various L, D, and D,L-enantiomers of the compound. In certain embodiments, the cysteine compound is N-acetyl-L-cysteine. In certain embodiments, the cysteine compound is N-acetyl-D-cysteine. In certain embodiments, the cysteine compound is N-acetyl-DL-cysteine. In certain embodiments, the cysteine compound is N-Fmoc-cysteine. In certain embodiments, the cysteine compound is N-acetyl-L-cysteine.

In certain embodiments, the non-aqueous solvent is propanol (including isopropanol). Other suitable solvents include ethanol, methanol, butanol (including isobutanol), dioxane, dimethoxyethane, bis(2-methoxyethyl)ether, octanol, t-butyl alcohol, or tetrahydrofuran (“THF”).

The suspension or slurry is heated to a temperature below the boiling point of the solvent. It is believed that heating the slurry to a temperature above room temperature minimizes the formation of undesirable side products, and thus provides for a product having high purity. In certain embodiments, the reaction temperature for each solvent will generally be from about 40° C. to about the boiling point of the solvent. For example, when using isopropyl alcohol, the solution is heated to about 80° C.-85° C. Using generally higher temperature favors complete reaction of the desired end product more quickly, efficiently and completely.

In particular, it has been found that when the reaction mixture and subsequent reactions are undertaken at a temperature of 40° C. or lower, a by-product is formed which has physicochemical properties (e.g. polarity) similar to the desired end product and which is very difficult to separate, particularly at production scale levels. Indeed, at less than 40° C., this impurity was discovered at levels approaching 2%.

Next, an approximately equimolar amount of an activated (halogenated) lipid is added slowly to the suspension or slurry, until the cysteine compound is consumed. In certain embodiments, the activated lipid is farnesyl bromide. In certain embodiments, the activated lipid is farnesyl chloride. In certain embodiments, the activated lipid is phytyl bromide. In certain embodiments, the activated lipid is phytyl chloride. In certain embodiments, the activated lipid is geranyl bromide. In certain embodiments, the activated lipid is geranyl chloride. In certain embodiments, the activated lipid is geranyl geranyl bromide. In certain embodiments, the activated lipid is geranyl geranyl chloride. In certain embodiments, the reaction mixture is stirred, and the consumption of the cysteine compound is monitored through regular sampling and analysis.

In certain embodiments, the rate of addition is matched to the rate of formation of the product to minimize the amount of impurities and to increase the overall reaction yield. This rate depends, inter alia, on the temperature at which the reaction is run and the solvent used. In certain embodiments, an excess of activated lipid is added to the reaction vessel to ensure complete consumption of the cysteine compound. By ensuring the complete consumption of odiferous starting materials, such as N-acetyl-cysteine, the odor of the final product is significantly reduced.

The reaction product is cooled and quenched with water to hydrolyze any unreacted activated lipid. The aqueous solution is washed at least once with a non-polar solvent, to remove non-polar impurities. In certain embodiments, the non-polar solvent is hexane. Other suitable non-polar solvents include heptane, pentane, benzene, toluene, diethyl ether, chloroform, ethyl acetate and/or combinations thereof.

In certain embodiments, AFC is prepared according to Scheme 4 set forth below.

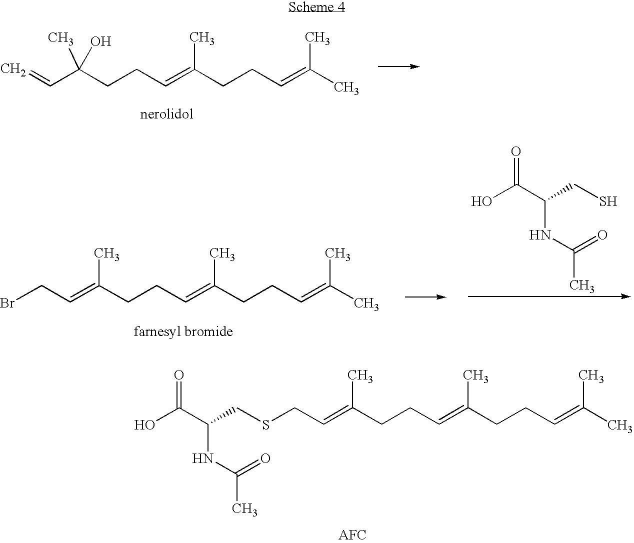

c. Purification

Once the reaction is complete, the crude product is purified by, for example, by performing acetonitrile wash(es) and acidification; conversation to a water-insoluble salt; and/or filtration with activated charcoal as shown in Scheme 5 below.

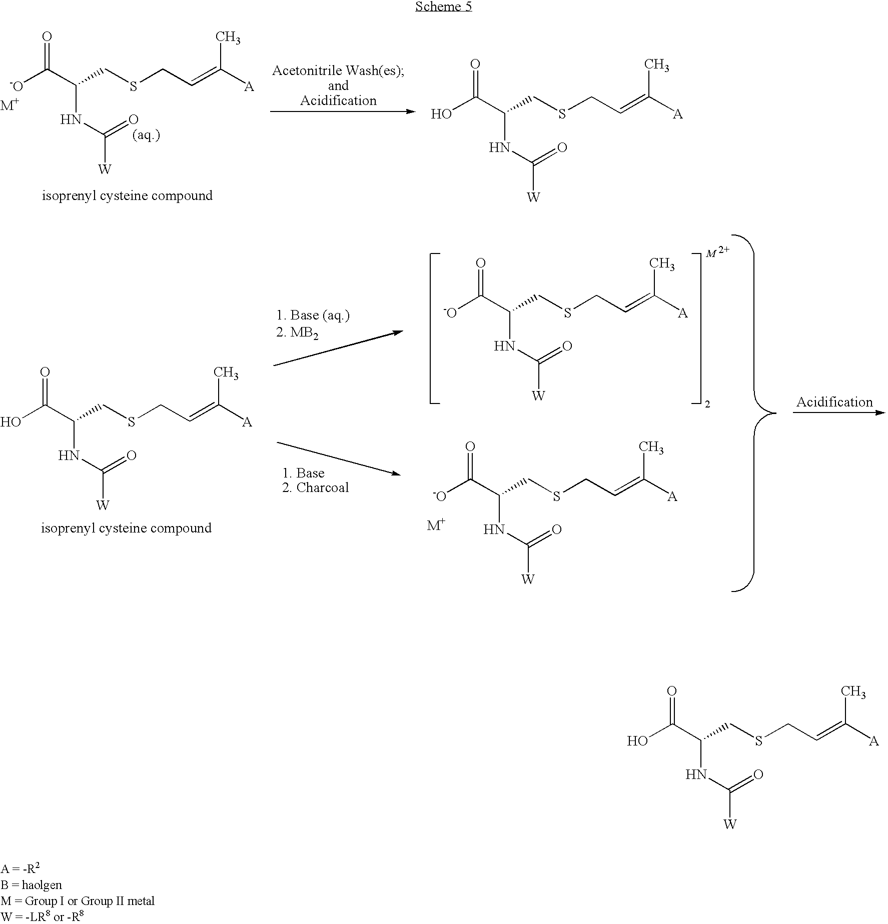

In certain embodiments, a method of purifying isoprenyl cysteine compounds is provided comprising steps of: (i) contacting a concentrated reaction mixture containing an isoprenyl cysteine compound with acetonitrile wash to remove non-polar impurities from the reaction mixture; and (ii) adjusting pH of the reaction mixture using an aqueous acid, to obtain an isoprenyl cysteine compound in high yield and high purity.

In certain embodiments, a method of purifying N-acetyl-S-farnesyl-L-cysteine is provided comprising steps of: (i) contacting a concentrated reaction mixture containing N-acetyl-S-farnesyl-L-cysteine with acetonitrile wash to remove non-polar impurities from the reaction mixture; and (ii) adjusting pH of the reaction mixture using an aqueous acid, to obtain N-acetyl-S-farnesyl-L-cysteine in high yield and high purity.

In certain embodiments, at least one or more acetonitrile wash(es) are performed. In certain embodiments, at least one acetonitrile wash is performed. In certain embodiments, at least two acetonitrile washes are performed. In certain embodiments, at least three acetonitrile washes are performed. In certain embodiments, at least four acetonitrile washes are performed. In certain embodiments, at least five acetonitrile washes are performed. In certain embodiments, at least six acetonitrile washes are performed. In certain embodiments, at least seven acetonitrile washes are performed.

In certain embodiments, acetonitrile wash(es) comprise contacting the reaction mixture with acetonitrile and/or combinations of acetonitrile and water. In certain embodiments, combinations of acetonitrile and water have a ratio of acetonitrile:water in a range of from about 5:1 to about 7:1. In certain embodiments, combinations of acetonitrile and water have a ratio in a range of from about 5:1 to about 6:1. In certain embodiments, combinations of acetonitrile and water have a ratio of about 5.6:1.

In certain embodiments, N-acetyl-S-farnesyl-L-cysteine is obtained in a yield of at least 80%. In certain embodiments, N-acetyl-S-farnesyl-L-cysteine is obtained having a purity of at least 95%.

In certain embodiments, the pH is adjusted to from about 2 to about 5. In certain embodiments, the pH is adjusted to from about 2 to about 3. In certain embodiments, the pH is adjusted to about 2.5.

In certain embodiments, the aqueous acid selected from HCl, phosphoric acid, NH₄Cl, and/or combinations thereof. In certain embodiments, the aqueous acid is HCl. In certain embodiments, the aqueous acid is phosphoric acid.

The present invention provides methods of purifying isoprenyl cysteine compounds by for example, recovering an aqueous solution (e.g., from a wash) containing the isoprenyl cysteine compounds and acidifying the solution with an aqueous acid, causing the product to become insoluble in water. In certain embodiments, the aqueous acid is HCl. In certain embodiments, the aqueous acid is phosphoric acid. In certain embodiments, an organic solvent is used to extract the product isoprenyl cysteine compound from the aqueous solution. In certain embodiments, when a water miscible organic solvent (such as isopropanol) is used as the solvent in the coupling reaction a residual amount of this solvent remains in the product solution after the reaction is quenched with water. Additional water miscible organic solvent may also be added to the solution of isoprenyl cysteine compound. The presence of about 10% to about 30% of such water miscible organic solvent is found to further improve yield, and/or enhance product separation into a distinct oil phase. The product is recovered as an oil and dried to remove any residual solvent. In certain embodiments, the oil is dried under vacuum.

Alternatively or additionally, isoprenyl cysteine compounds can be purified in accordance with the present invention through a process involving the formation of a water-insoluble divalent metal salt of the product through addition of a salt, such as CaCl₂ SrCl₂, or MgCl₂ to the aqueous product solution. This method works optimally when the conditions are adjusted such that the insoluble salt forms a fine suspension of particles. This may be achieved, for example, by slowly adding a solution of CaCl₂ to a solution of the sodium salt of an isoprenyl cysteine compound (e.g., AFC) with vigorous stirring. The product may be washed with non-polar solvent as in the first method prior to the divalent metal salt formation, or may be washed with the non-polar solvent after the salt formation. Generally, the water-insoluble salt will be less dense than water (or a brine solution) so it may be washed and the excess water drained from below to remove water-soluble impurities, for example, decomposed N-acetyl-cysteine and/or decomposed N-acetyl-S-farnesyl-L-cysteine. It may also be centrifuged to achieve an effective separation from the aqueous solution.

The product isoprenyl cysteine compound may be stored in a solid divalent metal salt form, or converted to another form. For example, the metal salt may be soluble in ethanol, THF and/or similar organic solvents, so it may be dissolved and then converted back into an oil form by the addition of a dilute acid such as HCl which brings the organic solvent to a final concentration of about 10% to about 30% in water.

Alternatively or additionally, purification step(s) may be employed after quenching the reaction with water to remove impurities. In certain embodiments, such additional purification steps are performed before formation of an oil. In certain embodiments, such additional purification steps are performed before formation of a divalent insoluble salt. Such alternative or additional purification step(s) comprise contacting the product in an aqueous solution with activated carbon, or passing the product solution through a bed of activated carbon or through a cartridge containing activated carbon to further improve the purity of the final product. In particular, such treatment with activated carbon may be advantageous for removing or minimizing certain odiferous impurities, especially polar impurities. Such activated carbon treatment may be advantageous for removing or minimizing certain polymerized farnesyl bromide if present. It will be appreciated by one skilled in the art that the technique of treating with activated carbon may be useful not only as described above, i.e., after quenching a reaction mixture, but it may be useful, for example, to treat a reaction mixture containing AFC with activated carbon, or to perform an activated carbon treatment after salt formation.

### II. Isoprenyl Cysteine Compounds

Inventive methods described herein may be applied to any of a variety of isoprenyl cysteine compounds.

AFC, and many other isoprenyl cysteine compounds are characterized by an ability to reduce methylation of a protein having a carboxyl-terminal -CAAX motif, wherein C=cysteine, A=any aliphatic amino acid, and X=any amino acid. (See Rando, U.S. Pat. No. 5,202,456). The methylation reaction which is inhibited is part of a series of post-translational modifications involving the -CAAX motif. These modifications include polyisoprenylation of the cysteine of the -CAAX motif (on the sulfur), proteolysis of the carboxyl-terminal three amino acids (-AAX) and methylation of the carboxyl group of cysteine.

In certain embodiments, inventive methods described herein can be applied to the preparation of one or more isoprenyl cysteine compounds. Isoprenyl cysteine compounds as described herein include small molecule compounds that are structurally related to N-acetyl-S-farnesyl-L-cysteine.

For example, in some embodiments, an isoprenyl cysteine compound has the structure set forth in formula I or formula II.

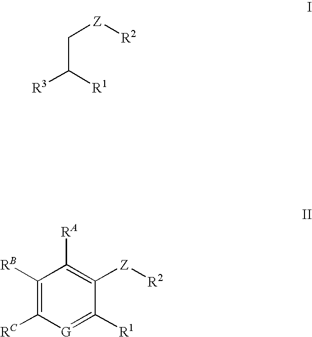

In some embodiments, an isoprenyl cysteine compound has the structure set forth in any of structures Ia-Ig.

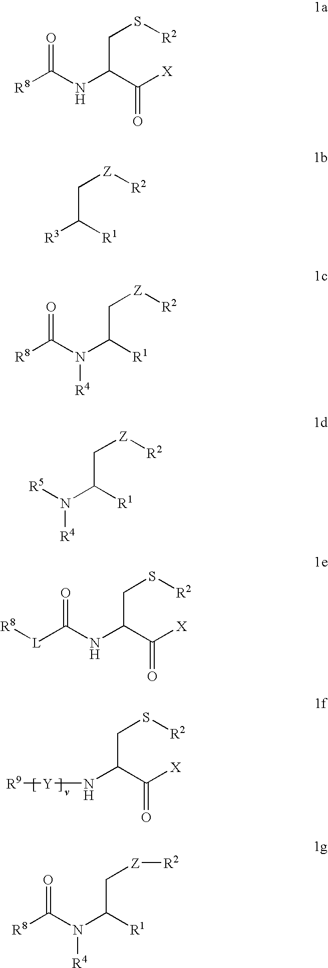

For example, according to the present invention, isoprenyl cysteine compounds include compounds of formula I:

According to the present invention, isoprenyl cysteine compounds include, for example, compounds of formula I:

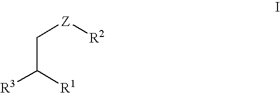

wherein:

R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl; or one of the following moieties:

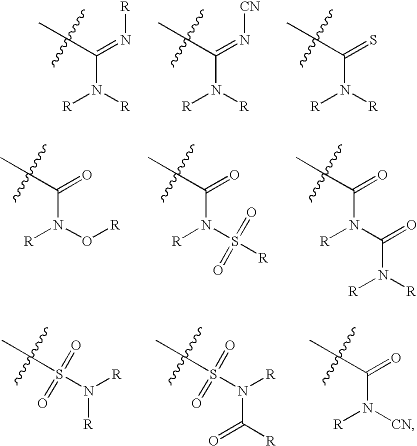

wherein each R is independently hydrogen or an optionally substituted group selected from C₁₋₆ aliphatic, C₁₋₆ heteroaliphatic, aryl, heteroaryl, or a cyclic radical;

R² is a substituted or unsubstituted, branched or unbranched C₁₀-C₂₅ aliphatic moiety;

R³ is —NH₂, a peptide, or —N(R⁴)(R⁵);

R⁴ is hydrogen or an optionally substituted group selected from C₁₋₆ aliphatic, C₁₋₆ heteroaliphatic, a cyclic radical, aryl or heteroaryl;

R⁵ is heteroaryl; —C(═N—R⁶)(R⁷), wherein R⁶ is selected from hydrogen, aliphatic, and —N(R)₂, and R⁷ is selected from hydrogen, aliphatic, aryl, cyano, and —SO₂R; or C(O)LR⁸, wherein L is a covalent bond or a bivalent, branched or unbranched, saturated or unsaturated, C₂-C₆ hydrocarbon chain wherein one or more methylene units of L is independently replaced by —O—, —S—, —NH—, —C(O)—, —C(═CH₂—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and R₈ is —R, —OR, —N(R)₂, a cyclic radical, aryl, heteroaryl, wherein each R is independently hydrogen or an optionally substituted group selected from C₁₋₆ aliphatic, C₁₋₆ heteroaliphatic, aryl, heteroaryl, or a cyclic radical; or a substituted or unsubstituted peptidic moiety; and

Z is —S—, —O—, —NH—, —Se—, —S(═O)—, —S(═N)—, —SO₂—, —Se(═O)—, or —SeO₂—.

In some embodiments, an isoprenyl cysteine compound has a structure depicted in formula Ia:

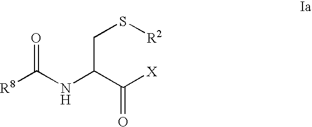

wherein R² is as defined herein;

X is —OH, halogen, methyl, —SH, —NH₂, or —N(R)₂, wherein R is hydrogen or C₁₋₃ alkyl; and

R⁸ is C₁₋₃ alkyl.

In some embodiments, an isoprenyl cysteine compound has a structure depicted in formula Ib:

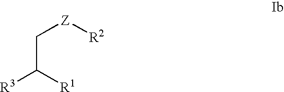

wherein

R¹ is —CO₂H, —CO₂R, —CONH₂, —NO₂, —PO₃H, —CN, or —SO₃H, where R is as defined herein;

R² is farnesyl, phytyl, geranylgeranyl, substituted farnesyl, substituted phytyl, or substituted geranylgeranyl; and

R³ is —NH₂ or a peptide.

In some embodiments, an isoprenyl cysteine compound has a structure depicted in formula Ic:

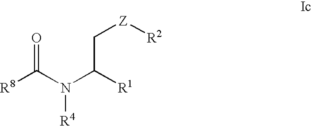

wherein R² and R⁸ are as described herein;

R¹ is substituted or unsubstituted heteroaryl, or one of the following moieties:

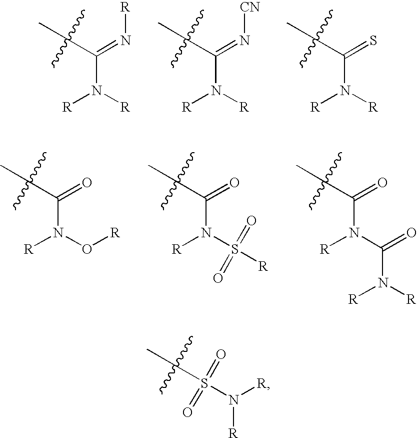

wherein
R is as described herein; and

Z is —S—, —O—, —Se—, —SO—, —SO₂—, or —NH—.

In some embodiments, an isoprenyl cysteine compound has a structure depicted in formula Id:

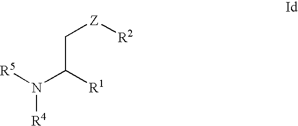

wherein R² and R⁴ are as described herein;

R¹ is substituted or unsubstituted heteroaryl, or one of the following moieties:

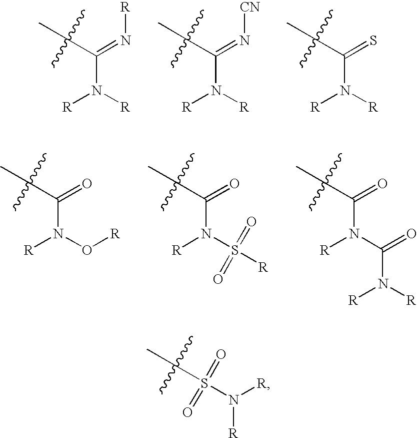

wherein
R is as described herein;

R⁵ is heteroaryl or —C(═NR⁶)(R⁷), where R⁶ and R⁷ are as described herein; and

Z is —S—, —O—, —Se—, —SO—, —SO₂—, or —NH—.

In some embodiments, an isoprenyl cysteine compound has a structure depicted in formula Ie:

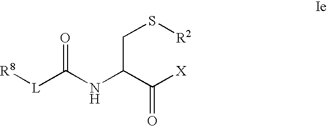

wherein R² is as described herein;

X is R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic;

L is a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and

R⁸ is hydrogen, —OH or —OR, wherein each R is independently hydrogen or an optionally substituted group selected from C₁₋₆ aliphatic or C₁₋₆ heteroaliphatic.

In some embodiments, an isoprenyl cysteine compound has a structure depicted in formula If:

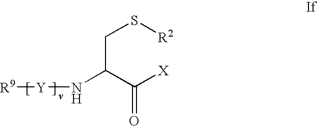

wherein

Y is a natural or unnatural amino acid;

v is an integer between 1 and 100, inclusive; and

R⁹ is hydrogen, a protecting group, or an optionally substituted group selected from C₁₋₆ aliphatic, C₁₋₆ heteroaliphatic, aryl or heteroaryl.

In some embodiments, an isoprenyl cysteine compound has a structure depicted in formula Ig:

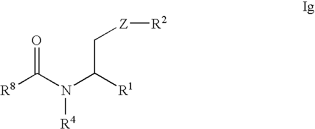

wherein:

Z is —S—, —O—, —Se—, —S(O)—, —SO₂—, or —NH—;

R¹ is a heteroaryl group, or a moiety selected from

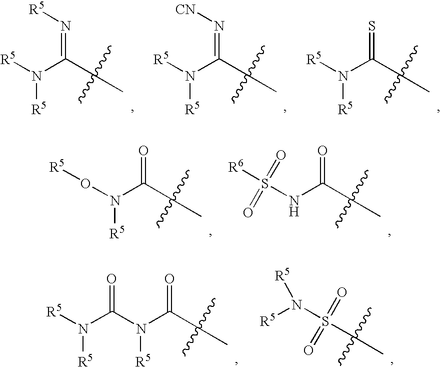

or

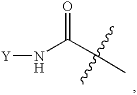

wherein at least one R⁵ group is H;

R⁵ is independently selected from H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, where R⁶ is optionally substituted with one or two R⁷ groups;

Y is selected from H, —NH₂, —OH, —NH-phenyl, —NHC(O)CH₃, —NHCH₃, or —(C₁-C₈)alkyl;

R² is an aliphatic group substituted with one or more R⁷ groups;

R⁸ is alkoxy, aminoalkyl, alkyl, aryl, alkenyl, alkynyl, or a cyclic radical, where R⁸ is optionally substituted with one or two R⁷ groups;

R⁴ is H, alkyl, aryl, alkenyl, alkynyl, or a cyclic radical, where R⁴ is optionally substituted with one or two R⁷ groups; and

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC (═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), ═OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(—O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

In some embodiments of any of the foregoing structures I and Ia-Ig, R¹ is an optionally substituted heteroaryl moiety of one of the formulae:

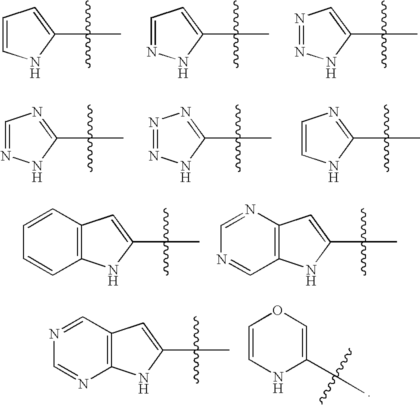

In some embodiments, R¹ is —CO₂H.

In some embodiments of any of the foregoing structures I and Ia-Ig, R² is a farnesyl group.

In some embodiments of any of the foregoing structures I and Ia-Ig, R³ is —NHCOCH₃.

In some embodiments of any of the foregoing structures I and Ia-Ig, Z is —S.

In some embodiments of any of the foregoing structures I and Ia-Ig, X is —OH.

In some embodiments, an isoprenyl cysteine compound has a structure depicted in formula II:

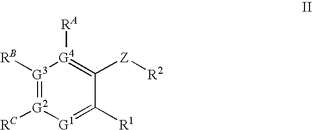

wherein each of G¹, G², G³, and G⁴ is N or CR^{D};

Z is S, O, Se, SO, SO₂, or NH;

R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl; or one of the following moieties:

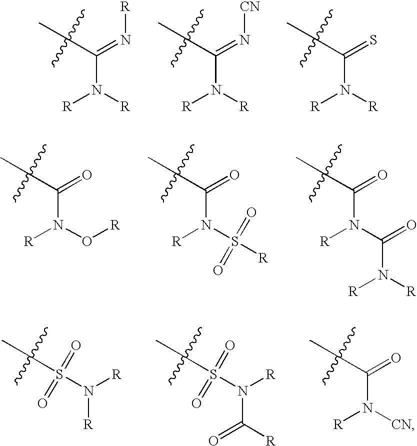

wherein each R is independently hydrogen or an optionally substituted group selected from C₁₋₆ aliphatic, C₁₋₆ heteroaliphatic, aryl, heteroaryl, or a cyclic radical;

R² is an optionally substituted aliphatic group;

R^{A}, R^{B}, R^{C}, and R^{D} are independently H, —NO₂, —OR¹⁰, halogen, alkylN(R¹⁰)₂, —N(R¹⁰)₂, —C(═O)R¹⁰, —C(═O)OR¹⁰, —S(R¹⁰), azido, —S—C≡N, alkyl, aryl, alkenyl, alkynyl, or a cyclic radical, wherein R^{A}, R^{B}, R^{C}, and R^{D} are further optionally substituted;

R¹⁰ is H, alkyl, aryl, alkenyl, alkynyl, or a cyclic radical, wherein R¹⁰ is further optionally substituted.

In some embodiments, at least one of G¹, G², G³, and G⁴ is N; in some embodiments, at least two of G¹, G², G³, and G⁴ are N; in some embodiments, at least three of G¹, G², G³, and G⁴ are N; in some embodiments, at least four of G¹, G², G³, and G⁴ are N. In some embodiments, G¹ is N. In some embodiments, G¹ is N and at least one of G², G³, and G⁴ is N.

### EXAMPLES

As depicted in the Examples below, in certain exemplary embodiments, compounds are prepared according to the following general procedures. It will be appreciated that, although the general methods depict the preparation of certain compounds of the present invention, the following general methods, and other methods known to one of ordinary skill in the art, can be applied to all classes, subclasses and species of each of these compounds, disclosed herein.

Additional background information and compounds utilized as starting materials may be prepared according to methods known in the art or prepared by the methods disclosed in WO04/020374 (Mero et al.); Stimmel et al., Evidence for an S-farnesylcysteine Methyl Ester at the Carboxyl Terminus of the Saccharomyces cerevisiae RAS2 protein, Biochemistry 29:9651-9659, 1990; Volker, C. et al., S-Farnesylcysteine Methyltransferase in Bovine Brain, Methods 1:283-287, 1990; Brown et al., Prenylated Proteins. A Convenient Synthesis of Farnesyl Cysteinyl Thioethers, J. Am. Chem. Soc., 113:3176-3177, 1991; 1991; Yang et al., 1991. Efficient Method for Regioselective Isoprenylation of Cysteine Thiols in Unprotected Peptides, J. Am. Chem. Soc. 113:3177-3178; Tan et al., Identifying the Recognition Unit for G protein Methylation, J. Biol. Chem. 266:10719-10722, 1991; and Perrey et al., F.M. 2001. An Improved Method for Cysteine Alkylation, Tetrahedron Lett. 42:1859-1861, the disclosure of which is incorporated by reference herein.

### Example 1

### 2-Step Preparation of N-acetyl-S-farnesyl-L-cysteine

### Step 1: Preparation of Farnesyl Bromide with Triethylamine in Toluene

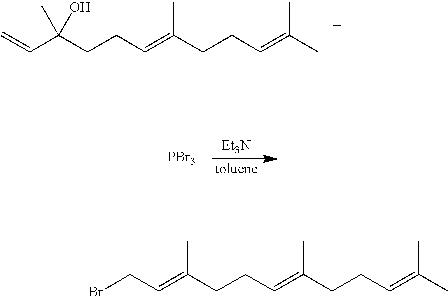

| | | | Den- | | | |
|---|---|---|---|---|---|---|
| | | Vol- | sity | | | |
| | | ume | (g/ | MW | | Equiva- |
| Material | Mass (g) | (mL) | mL) | (g/mole) | Moles | lents |
| Nerolidol | 20 | 22.9 | 0.875 | 222.37 | 0.09 | 1 |
| Triethylamine | 1.3 | 1.8 | 0.726 | 101.19 | 0.013 | 0.15 |
| Toluene (pot) | 30 | 34.7 | 0.865 | 92.14 | 0.326 | na |
| Phosphorus | 8.9 | 3.1 | 2.85 | 270.7 | 0.033 | 0.37 |
| tribromide | | | | | | |
| Toluene | 7 | 8.1 | 0.865 | 92.14 | 0.076 | na |
| (wt PBr3) | | | | | | |

A 250 mL flask with bottom spout, paddle stirrer, thermocouple, and 125 mL dropping funnel was purged with nitrogen for 20 minutes and charged with the nerolidol, toluene, and triethylamine. The jacket was cooled to −5° C. with a glycol/water chiller. The dropping funnel was charged with the phosphorus tribromide and additional toluene. The PBr₃/toluene solution was added drop wise over a 25 minute period:

| Time | Pot Temperature | Solution |
|---|---|---|
| (min) | (° C.) | (mL) |
| 0 | 3.3 | 12 |
| 5 | 6 | 11 |
| 10 | 6.6 | 10 |
| 15 | 3.6 | 8 |
| 21 | 0.1 | 4 |
| 25 | −0.4 | 0 |

The jacket was then warmed to 23° C. After 15 minutes, the pot temperature was 20° C. and the first reaction sample was taken. After 55 minutes, brown “goo” was noticed on the sides and bottom of the flask. A second reaction sample was taken. There was no difference between the two HPLC samples, nor was there any evidence of unreacted nerolidol. Deionized water (41.1 g) was added and a slight (1.5° C.) exotherm was observed. The mixture was agitated for 30 minutes during the time which the brown goo became dissolved. The mixture was allowed to settle for 30 minutes. A clean split of 45.0 g of cloudy orange/brown lower aqueous layer was split off (density=1.034 g/mL). Brine (39.5 g, 15% solution) was added and agitated for 15 minutes and then allowed to settle for 30 minutes. A clean split of 39.6 g of nearly clear, colorless lower aqueous layer (density=1.100 g/mL) was split off from 59.8 g of clear, yellow organic layer. The bulk of the toluene was removed by rotary evaporation at 15 mm vacuum and 45° C. until no more solvent came over. The material was then put on an oil pump vacuum (typical vacuum <0.1 mm) for 75 minutes at room temperature. A total of 24.6 g of a yellow liquid was obtained (96% yield assuming pure farnesyl bromide).

### Step 2: Preparation of N-acetyl-S-farnesyl-L-cysteine and Hexane Wash

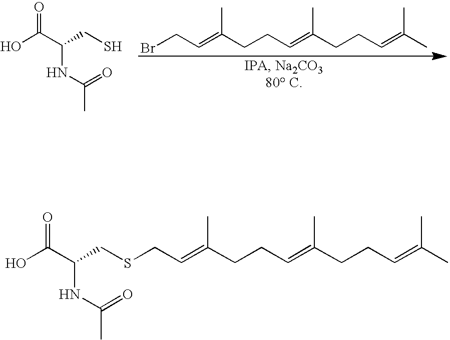

| Reagent | | | | | | |
|---|---|---|---|---|---|---|
| No. | Compound | MW | Amount | | Mol. | Equivalents |
| 1 | N-acetyl-L-cysteine | 163.2 | 320 | g | 1.96 | 1.0 |
| 2 | farnesyl bromide | 285.3 | 620 | ml, | 2.05 | 1.05 |
| | | | (90%) | | | |
| | | | (d. 1.052) | | | |
| 3 | Na₂CO₃ | 124 | 316.0 | g | 2.55 | 1.6 |
| 4 | isopropanol | — | 1.05 | lit | — | — |

A 2 L, three-necked round-bottomed flask, was equipped with mechanical stirrer, 250 mL pressure equalizing dropping funnel and thermometer. The flask was placed in heating mantle and charged with N-acetyl cysteine (320 g, 1.96 mol), Na₂CO₃ (316 g, 2.55 mol) followed by isopropanol (1.05 L). The stirred suspension was heated to 80° C. and then farnesyl bromide (620 mL, 2.05 mol, the rate of addition at 40 mL/h initial 2 h and then increased to 60 mL/h remaining time) was added drop wise through the dropping funnel over a period of 11 h. The internal temperature of the reaction mixture was maintained between 78° C. to 82° C. The reaction mixture was monitored by TLC/HPLC for disappearance of starting material. At the end of the farnesyl bromide addition, the HPLC showed completion of the reaction.

The reaction mixture was cooled to room temperature (between about 20° C. and about 26° C.) and then diluted with water (1800 mL), and hexane (1500 mL). The resulting mixture was stirred at this temperature for about 30 minutes, and then transferred to a separatory funnel, and the organic phase was separated. The aqueous phase was washed several times with hexane (3 times, 2 liters each) to remove the non-polar impurities completely. The aqueous phase was adjusted to a pH of ˜2 by addition of aqueous HCl. The mixture was transferred to a separatory funnel, the AFC (top layer) was separated, the isopropyl alcohol removed in vacuo and dried under high vacuum for three days. Yield: 648 g, 90% (purity >99%).

### Example 2

### Preparation of N-acetyl-S-farnesyl-L-cysteine using Calcium Salt

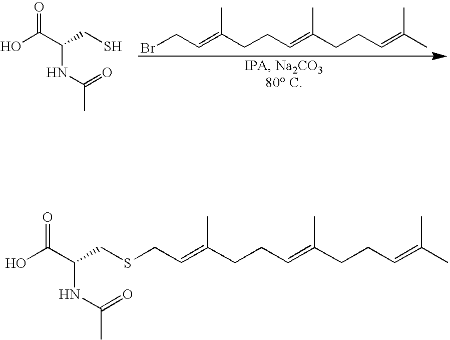

| Reagent | | | | | | |
|---|---|---|---|---|---|---|
| No. | Compound | MW | Amount | | Mol | Equivalents |
| 1 | N-acetyl-L-cysteine | 163.2 | 150 | g | 0.92 | 1.25 |
| 2 | farnesyl bromide | 285.3 | 199 | ml | 0.74 | 1.0 |
| 3 | Na₂CO₃ | 124 | 146.8 | g | 1.18 | 1.6 |
| 4 | isopropanol | — | 500 | ml | — | — |

A 2 L, three-necked round-bottomed flask was equipped with mechanical stirrer, 125 mL pressure equalizing dropping funnel and thermometer. The flask was placed in heating mantle and charged with N-acetyl cysteine (150 g, 0.92 mol), sodium carbonate (146.8 g, 1.18 mol) followed by isopropanol (500 mL) as solvent. The stirred suspension was heated to 80° C. and then farnesyl bromide (199 mL, 0.45 mol, rate of addition at 10 mL/h initial 2 h and then increased to 25 mL/h during the remaining time) was added drop wise through the dropping funnel over a period of 9 hours. The internal temperature of the reaction mixture was maintained between about 80° C. to about 85° C. At the end of farnesyl bromide addition, the HPLC showed reaction was complete. The reaction mixture was cooled to room temperature and then diluted with water (900 mL) and hexane (600 mL).

The resulting mixture was stirred at this temperature for 15 min, and then transferred to a separatory funnel; the organic phase was separated; and the aqueous phase washed several times with hexane (4 times, 600 mL each) to remove the non-polar impurities. To the stirred aqueous phase, calcium chloride (145 g in 150 mL water) was added to precipitate the AFC as the calcium salt, and then diluted with more water (600 mL). The resulting mixture was cooled in the refrigerator overnight to allow setting or the sticky precipitate in the top and water in the bottom. The water phase was slowly decanted under vacuum. The sticky precipitate was washed with acetonitrile (300 mL), hexane (400 mL) and removed the solvent similarly, the sticky precipitate again taken in to water (1000 mL) left in the refrigerator overnight. The next day, the water was decanted slowly using vacuum and a water wash was repeated several times (600 mL×3).

The calcium salt of AFC was suspended in 800 mL of THF and adjusted pH to 2 by addition of aqueous HCl. The solvent was removed in vacuo and then transferred to a separatory funnel. The AFC (top layer) was separated and dried under high vacuum to yield 221 g (82%). ˜99% pure by HPLC.

### Example 3

### Preparation of Geranyl Geranyl Bromide from Geranyl Linalool

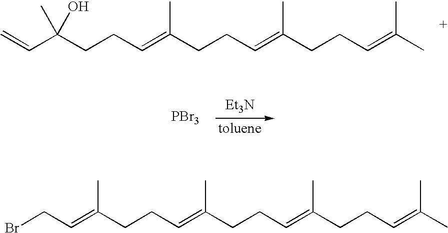

| Reagent | MW | Amount | | Density | Mmoles | Equivalents |
|---|---|---|---|---|---|---|
| geranyl linalool | 290.48 | 5.0 | ml | 0.88 | 15.5 | 1.0 |
| phosphorus | 270.69 | 711.8 | μl | 2.88 | 7.57 | 0.5 |
| tribromide (PBr₃) | | | | | | |
| toluene | — | 15 | ml | — | — | 1 ml/mmol |
| triethylamine | 101.19 | 211.1 | μl | 0.726 | 1.51 | 0.1 |
| (Et₃N) | | | | | | |

Geranyl linalool and Et₃N was mixed by magnetic stirring and cooled in a CH₃CN/dry ice bath. PBr₃ and toluene was mixed in an addition funnel and added drop wise over the course of 1 hour. The reaction mixture was removed from the dry ice bath and left stirring while it warmed to room temperature. The reaction was quenched by the drop wise addition of 25 mL of water over 1 hour. The product was extracted 3 times with 25 mL EtOAc. Then the combined organic phase was returned to the separation funnel and washed twice with 25 mL saturated NaHO₃, using brine to break up the emulsion. The reaction was allowed to stand overnight to resolve the phases, dried over Na₂SO₄ and the solvent was removed in vacuo.

### Example 4

### Preparation of Farnesyl Bromide from Farnesol

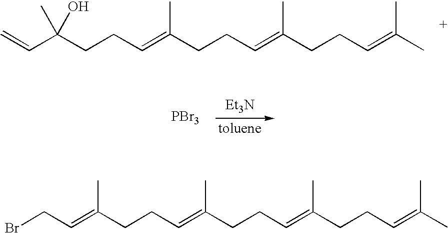

| Reagent | MW | Amount | | Density | Moles | Equivalents |
|---|---|---|---|---|---|---|
| Farnesol | 222.37 | 566.5 | ml | 0.886 | 2.25 | 1.0 |
| Phosphorus | 270.69 | 79.1 | ml | 2.85 | 0.833 | 0.37 |
| tribromide (PBr₃) | | | | | | |
| Toluene | — | 800 | ml | — | — | — |
| triethylamine | 101.19 | 47.1 | ml | 0.726 | 0.34 | 0.15 |
| (TEA) | | | | | | |

A 5 L, 3-necked RB flask equipped with a mechanical stirrer, thermometer, and dropping funnel was charged with farnesol, toluene, and Et₃N. The mixture was cooled to −5° C. under argon atmosphere. The PBr₃ was mixed with 210 mL additional toluene and added drop wise over a period of 2 hours. After the addition was complete, the reaction mixture was allowed to come to room temperature and a sample was taken for analysis. HPLC analysis at 214 nm using a reversed-phase C18 column showed completion of the reaction. The reaction was stirred at room temperature for 1 hour. The reaction was then quenched with 1 liter cold water, stirred for 30 minutes, and then transferred to a separatory funnel. The organic phase was separated. The aqueous phase was extracted with an additional 500 mL of toluene and both organic (toluene) phases were combined. The combined organic phase was washed thoroughly was water (twice with 1 liter) and brine (twice with 0.5 liter). The organic layer was dried over Na₂SO₄, the solvent removed in vacuo for 6 hours and dried under high vacuum. The yield was 628 g (93% pure by HPLC), which corresponds to ˜91% yield.

### Example 5

### Purification of AFC Using Calcium Salt

AFC was prepared using 16.3 g (0.1 mol) of N-acetyl-L-cysteine. The AFC reaction mixture was poured into aqueous NaOH (50 mL of 2N solution), chilled with ice, and then transferred to a separation funnel. Non-polar impurities were removed by washing with hexanes (3×100 mL). The aqueous solution of sodium salt of AFC was treated with CaCl₂ solution (15 g; 0.1 mol in 10 mL of water) to precipitate calcium salt of AFC as off-white solid (with appearance of cottage cheese). This solid was washed with water (2×100 mL), most of the water was drained then calcium salt of AFC was centrifuged and suspended in THF (40° C., 200 mL) and acidified to a pH of about 2.5 by addition of 10% HCl. The THF was removed in vacuo, and the AFC came out as a thick oil easily collected from separation funnel. The oil was further dried on a vacuum pump to afford a thick yellowish oil (21.3 g, 58% yield); 96% purity (by HPLC).

### Example 6

### Preparation of N-acetyl-S-farnesyl-L-cysteine and Acetonitrile Wash Purification

| Com- | | | | | |
|---|---|---|---|---|---|
| pound | Compound | Molecular | | | Equiv- |
| No. | Name | Weight | Amount | Mmol | alents |
| 1 | N-acetyl-L- | 163.2 | 100.0 g | 612.75 | 1.0 |
| | cysteine | | | | |
| 2 | farnesyl | 285.3 | 206.3 g | 723.10 | 1.18 |
| | bromide | | (d.1.052; | | |
| | | | 204 ml, 96%) | | |
| 3 | Na₂CO₃ | 124 | 99.0 g | 798.39 | 1.3 |
| 4 | isopropanol | — | 340 mL | — | — |

A 2 L three necked round-bottomed flask was equipped with mechanical stirrer, 125 mL pressure equalizing dropping funnel and thermometer. The flask was placed in heating mantle and charged with N-acetyl cysteine (100 g, 612.75 mmol), isopropanol (340 mL) and Na₂CO₃ (99 g, 798.39 mmol). The stirred suspension was heated to 82° C. and then farnesyl bromide (204 mL, 96%, 723.1 mmol) was added drop wise through a dropping funnel over a period of 9 hours (the internal temperature of the reaction mixture was maintained between 80 to 82° C.). The reaction mixture was monitored by TLC/HPLC disappearance of starting materials N-acetyl cysteine and farnesyl bromide. At the end of farnesyl bromide addition, the HPLC showed completion of the reaction.

The reaction mixture was cooled and transferred to a 1000 mL RB flask, the solvent was removed by using rotary evaporator, the concentrated mixture (about 15% of IPA is left) was washed with acetonitrile (160 mL), the mixture was mixed with mechanical stirrer at 65° C. for 45 min, and then cooled in the freezer for about 1 hour and then decanted away the acetonitrile layer, followed by four additional washes of a mixture of CH₃CN/H₂O (each wash volume was 200 mL, having a mixture of 170 mL CH₃CN/30 mL H₂O), stirred at 65° C. for 45 min, cooled in the freezer for 1 hour, and then decanted acetonitrile, and removed non-polar impurities. NMR showed over 97% pure per integration. The clean reaction mixture was dissolved in mixture of H₂O/CH₃CN (300 mL, 2:1) and adjusted pH to 2.5 by addition of 20% HCl. The mixture was transferred to a separatory funnel, the AFC (top layer) was separated, the acetonitrile was removed in vacuo and the product was dried under high vacuum for three days. % Yield: 186.8 g, about 83% (purity >99% by HPLC).

### Example 7

### Additional Purification of AFC Sodium Salt Using Activated Carbon

To AFC (117.7 g) was added a solution of sodium hydroxide (2 M, 150 mL, pH adjusted to ˜10.0) to form an AFC sodium salt. This salt mixture was further diluted with water (450 mL), activated carbon (17 g) was added, and the resulting mixture heated to ˜90° C. for 3.5 hours. The hot mixture was passed through a celite bed, washed thoroughly with water, and acidified to a pH of about ˜2.5 by addition of HCl (to result in AFC). The AFC was extracted into ethyl acetate, dried over Na₂SO₄, concentrated under reduced pressure, and dried under high vac for 20 hours to yield pure, odorless AFC (105.8 g).

### EQUIVALENTS

Those skilled in the art will recognize, or be able to ascertain using no more than routine experimentation, that while the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or sub range within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

We claim:

1. A method of purifying N-acetyl-S-farnesyl-L-cysteine comprising steps of:
(i) contacting a concentrated reaction mixture containing N-acetyl-S-farnesyl-L-cysteine with acetonitrile wash to remove non-polar impurities from the reaction mixture; and
(ii) adjusting pH of the reaction mixture using an aqueous acid, to obtain N-acetyl-S-farnesyl-L-cysteine in high yield and high purity.

2. The method according to claim 1, further comprising obtaining the N-acetyl-S-farnesyl-L-cysteine in a yield of at least 80%.

3. The method according to claim 1, further comprising obtaining the N-acetyl-S-farnesyl-L-cysteine with a purity of at least 95%.

4. The method according to claim 1 wherein the pH is adjusted to from about 2 to about 5.

5. The method according to claim 4 wherein the pH is adjusted to from about 2 to about 3.

6. The method according to claim 5 wherein the pH is adjusted to about 2.5.

7. The method according to claim 1 wherein the aqueous acid selected from HCl, phosphoric acid, NH₄Cl, and/or combinations thereof.

8. The method according to claim 1 wherein the aqueous acid is HCl.

9. The method according to claim 1, wherein the acetonitrile wash comprises contacting the reaction mixture with acetonitrile and/or combinations of acetonitrile and water.

10. The method according to claim 9 wherein combinations of acetonitrile and water have a ratio of acetonitrile:water in a range of from about 5:1 to about 7:1.

11. The method according to claim 10 wherein the combination of acetonitrile and water has a ratio in a range of from about 5:1 to about 6:1.

12. The method according to claim 11 wherein the combination of acetonitrile and water has a ratio of about 5.6:1.

13. An anhydrous method of preparing N-acetyl-S-farnesyl-L-cysteine comprising steps of: (a) coupling farnesyl bromide with N-acetyl-cysteine in a non-aqueous solvent at a molar concentration ranging from about 0.1 M to about 5 M in the presence of 0.5-1.5 equivalents of an inorganic base at a temperature of about 80° C.-85° C. to yield a reaction mixture containing N-acetyl-S-farnesyl-L-cysteine; and (b) purifying a concentrated reaction mixture containing N-acetyl-S-farnesyl-L-cysteine comprising steps of: (i) contacting the reaction mixture with acetonitrile wash to remove non-polar impurities from the reaction mixture; and (ii) adjusting pH of the reaction mixture using an aqueous acid, to obtain the N-acetyl-S-farnesyl-L-cysteine with a purity of at least 95%.

14. The method according to claim 13, further comprising obtaining the N-acetyl-S-farnesyl-L-cysteine in a yield of at least 80%.

15. The method of claim 13, wherein the aqueous solvent is selected from propanol (e.g., isopropanol), ethanol, methanol, butanol (e.g., isobutanol), dioxane, dimethoxyethane, bis(2-methoxyethyl)ether, octanol, t-butyl alcohol, tetrahydrofuran (“THF”) and combinations thereof.

16. The method according to claim 13 wherein the temperature is about 80° C.

17. The method according to claim 13, wherein the inorganic base is Na₂CO₃.

18. An anhydrous method of preparing N-acetyl-S-farnesyl-L-cysteine comprising a step of coupling farnesyl bromide with N-acetyl-cysteine in isopropyl alcohol at a molar concentration ranging from about 0.1 M to about 5 M in the presence of 0.5-1.5 equivalents of an inorganic base at a temperature of about 80° C.-85° C., wherein the N-acetyl-S-farnesyl-L-cysteine has a purity of at least 95%.

19. The method according to claim 18 wherein the temperature is about 80° C.

20. The method according to claim 18, further comprising obtaining the N-acetyl-S-farnesyl-L-cysteine in a yield of at least 80%.

21. The method according to claim 18, wherein the inorganic base is Na₂CO₃.
